(19) United States
(12) Patent Application Publication
(10) Pub. No.: US 2024/0358788 A1
(43) Pub. Date: Oct. 31, 2024

(54) COMPOSITIONS AND METHODS FOR TREATING RADIATION DERMATITIS

(71) Applicants: April Brader, St. Petersburg Beach, FL (US); Joel Snook, St. Petersburg Beach, FL (US); Yohann Samarasinghe, La Jolla, CA (US); Samarth Aggarwal, San Diego, CA (US)

(72) Inventors: April Brader, St. Petersburg Beach, FL (US); Joel Snook, St. Petersburg Beach, FL (US); Yohann Samarasinghe, La Jolla, CA (US); Samarth Aggarwal, San Diego, CA (US)

(21) Appl. No.: 18/306,650

(22) Filed: Apr. 25, 2023

(51) Int. Cl. A61K 36/704; A61K 9/00; A61K 35/648; A61K 36/286; A61K 36/30; A61K 36/489; A61K 36/68; A61K 36/718; A61K 36/756; A61K 47/10; A61K 47/26; A61K 47/44; A61P 17/18

(52) U.S. Cl. CPC A61K 36/704; A61K 9/0014; A61K 35/648; A61K 36/286; A61K 36/30; A61K 36/489; A61K 36/68; A61K 36/718; A61K 36/756; A61K 47/10; A61K 47/26; A61K 47/44; A61P 17/18

(57) ABSTRACT

A composition having two or more organic components selected from Japanese knotweed, Cortex Dictamni, Rhizoma Coptidis, Radix Sophorae Flavescentis, Cortex Phellodendri, Calendula officinalis, Plantago lanceolata, Radix Arnebiae, Flos Carthami, and Scolopendra, and a method of using the composition to treat the skin condition of a subject in need thereof, are disclosed.

### FIELD

The present disclosure generally relates to compositions for topical administration, or topical compositions, methods of preparing the topical compositions, and methods of using the topical compositions to treat a subject in need thereof; and, more particularly, to topical compositions having one or more herbal components and one or more animal components (collectively, organic components), methods of preparing the topical compositions with the organic components, and methods of treating a subject having a skin condition with the topical compositions with the organic components.

### BACKGROUND

Dermatitis is inflammation of the skin. Dermatitis can vary in duration from short bouts to long-term cases and can involve a small area of skin or cover the entire body. There are several types of dermatitis. Radiation dermatitis, for example, is associated with prolonged exposure to ionizing radiation and occurs in patients receiving radiation therapy. The condition manifests itself in localized red patches of skin, or burns. There are no known cures for radiation dermatitis, and treatment goals usually focus on controlling and minimizing the symptoms. Additional therapeutic compositions for the treatment of dermatitis are needed.

### SUMMARY

In various embodiments, a composition having two or more organic components selected from the group consisting of: Japanese knotweed, Cortex Dictamni, Rhizoma Coptidis, Radix Sophorae Flavescentis, Cortex Phellodendri, Calendula officinalis, Plantago lanceolata,Radix Arnebiae, Flos Carthami, and Scolopendra, is provided, wherein each of the organic components is included in an amount ranging from about 0.0001% to about 10%, or from about 0.001% to about 8%, or from about 0.01% to about 5%, or from about 0.1% to about 1%.

In some embodiments, the composition includes each of the following organic components: Japanese knotweed, Cortex Dictamni, Rhizoma Coptidis, Radix Sophorae Flavescentis, Cortex Phellodendri, Calendula officinalis, Plantago lanceolata, Radix Arnebiae, Flos Carthami, and Scolopendra.

In some embodiments, the composition further includes coco glucoside, glycerin, water, and/or ethanol. In some embodiments, the composition further includes beeswax, coconut oil, and/or stearic acid. In some embodiments, the composition further includes coco glucoside, glycerin, water, ethanol, beeswax, coconut oil, and stearic acid. In some embodiments, the composition further includes coco glucoside, glycerin, beeswax, coconut oil, and stearic acid.

In some embodiments, the composition includes about 3% to about 6% of the organic components (collectively); about 3% to about 6% of coco glucoside; about 20% to about 25% of glycerin; about 4% to about 8% of water; and/or about 4% to about 8% of ethanol; wherein the respective amounts are based on weight.

In some embodiments, the composition includes: 3% to 7% of the organic components; 3% to 7% of coco glucoside; 20% to 25% of glycerin; 4% to 9% of water; and/or 4% to 9% of ethanol; wherein the respective amounts are based on weight.

In various embodiments, a topical composition for treating radiation dermatitis, having a plurality of organic components selected from: Japanese knotweed, Cortex Dictamni, Rhizoma Coptidis, Radix Sophorae Flavescentis, Cortex Phellodendri, Calendula officinalis, Plantago lanceolata, Radix Arnebiae, and Flos Carthami, is described, wherein each of the organic components is in an amount ranging from about 0.0001% to about 10%, or from about 0.001% to about 8%, or from about 0.01% to about 5%, or from about 0.1% to about 1%.

In some embodiments, the topical composition further comprises coco glucoside, glycerin, water, and/or ethanol. In some embodiments, the topical composition further comprises coco glucoside and glycerin. In some embodiments, the topical composition further comprises coco glucoside, glycerin, water, and ethanol.

In some embodiments, the topical composition further includes beeswax, coconut oil, and/or stearic acid. In some embodiments, the topical composition further comprises beeswax, coconut oil, stearic acid, coco glucoside and glycerin. In some embodiments, the topical composition further comprises beeswax, coconut oil, stearic acid, coco glucoside, glycerin, water, and ethanol.

In some embodiments, the topical composition includes about 3% to about 7% of the organic components; about 3% to about 7% of coco glucoside; about 20% to about 25% of glycerin; about 4% to about 9% of water; and about 4% to about 9% of ethanol; wherein the respective amounts are based on weight.

In some embodiments, the topical composition includes: 4% to 5% of the organic components; 4% to 5% of coco glucoside; 22% to 23% of glycerin; 6% to 7% of water; and 6% to 7% of ethanol; wherein the respective amounts are based on weight.

In some embodiments, the topical composition includes about 3% to about 20% of the organic components contained in glycerin; about 3% to about 20% of coco glucoside; about 10% to about 40% beeswax; about 10% to about 40% coconut oil; and about 2% to about 20% stearic acid; wherein the respective amounts are based on weight.

In some embodiments, the topical composition includes about 10% to about 15% of the organic components contained in glycerin; about 10% to about 15% of coco glucoside; about 25% to about 35% beeswax; about 28% to about 38% coconut oil; and about 5% to about 15% stearic acid; wherein the respective amounts are based on weight.

In various embodiments, a method of treating a skin condition in a subject in need thereof is described herein. In such embodiments, the method includes the step of administering a topical composition to the subject, the topical composition including an organic mixture having two or more organic components selected from the following: Japanese knotweed, Cortex Dictamni, Rhizoma Coptidis, Radix Sophorae Flavescentis, Cortex Phellodendri, Calendula officinalis, Plantago lanceolata, Radix Arnebiae, Flos Carthami, and Scolopendra, wherein each of the organic components is in an amount ranging from about 0.0001% to about 10%, or from about 0.001% to about 8%, or from about 0.01% to about 5%, or from about 0.1% to about 1%.

In some embodiments of the method, the skin condition is dermatitis. In some embodiments of the method, the dermatitis is radiation dermatitis. In some embodiments of the method, the step of administering the topical composition to the subject occurs twice daily.

In some embodiments of the method, the topical composition includes each of the respective organic components.

In some embodiments of the method, further includes coco glucoside, glycerin, water, and/or ethanol. In some embodiments, the composition further includes beeswax, coconut oil, and/or stearic acid. In some embodiments, the composition further includes coco glucoside, glycerin, water, ethanol, beeswax, coconut oil, and stearic acid. In some embodiments, the composition further includes coco glucoside, glycerin, beeswax, coconut oil, and stearic acid.

In some embodiments of the method, the topical composition includes about 3% to about 6% of the organic mixture; about 3% to about 6% of coco glucoside; about 20% to about 25% of glycerin; about 4% to about 8% of water; and about 4% to about 8% of ethanol; wherein the respective amounts are based on weight.

In some embodiments of the method, the topical composition includes: 4% to 5% of the respective organic components; 4% to 5% of coco glucoside; 22% to 23% of glycerin; 6% to 7% of water; and 6% to 7% of ethanol; wherein the respective amounts are based on weight.

In some embodiments of the method, the topical composition includes: 4% to 5% of the respective organic components; 4% to 5% of coco glucoside; 22% to 23% of glycerin; 6% to 7% of water; 6% to 7% of ethanol; beeswax; coconut oil, and stearic acid; wherein the respective amounts are based on weight.

In some embodiments of the method, the topical composition includes about 3% to about 20% of the organic components contained in glycerin; about 3% to about 20% of coco glucoside; about 10% to about 40% beeswax; about 10% to about 40% coconut oil; and about 2% to about 20% stearic acid; wherein the respective amounts are based on weight.

In some embodiments of the method, the topical composition includes about 10% to about 15% of the organic components contained in glycerin; about 10% to about 15% of coco glucoside; about 25% to about 35% beeswax; about 28% to about 38% coconut oil; and about 5% to about 15% stearic acid; wherein the respective amounts are based on weight.

It is to be understood that both the foregoing general description and the following detailed description describe various embodiments and are intended to provide an overview or framework for understanding the nature and character of the claimed subject matter. The accompanying drawings are included to provide a further understanding of the various embodiments and are incorporated into and constitute a part of this specification. The drawings illustrate the various embodiments described herein and, together with the description, explain the principles and operations of the claimed subject matter.

### BRIEF DESCRIPTION OF THE DRAWINGS

A complete understanding of the present embodiments and the advantages and features thereof will be more readily understood by reference to the following detailed description, appended claims, and accompanying drawings, wherein:

FIG. 1 is a photograph of a subject who has severe radiation burns of his head and neck from radiation oncology treatments; and

FIG. 2 is a photograph of the subject after treatment with a topical composition described herein.

The drawings are not necessarily to scale, and certain features and certain views of the drawings may be shown exaggerated in scale or in schematic in the interest of clarity and conciseness.

### DETAILED DESCRIPTION

Reference will now be made in detail to the exemplary embodiment(s), examples of which is/are illustrated in the examples. Before describing the exemplary embodiments, it is noted the embodiments reside primarily in combinations of components and procedures related to the topical compositions and methods of making and using the topical compositions. As such, the composition and method components have been represented where appropriate, showing only those specific details that are pertinent to understanding the embodiments of the present disclosure so as not to obscure the disclosure with details that will be readily apparent to those of ordinary skill in the art having the benefit of the description herein. The specific details of the various embodiments described herein are used for demonstration purposes only, and no unnecessary limitations or inferences are to be understood therefrom.

In various embodiments, a composition for topical administration, or topical composition, is provided. In some embodiments, the topical composition may be used to treat one or more skin conditions or disorders, including, for example, dermatitis on the skin of a subject in need thereof. In some embodiments, the topical composition may be used to treat radiation dermatitis on the skin of a subject in need thereof. In various embodiments, a method of administering the topical composition to the skin of a subject in need thereof is provided. In some embodiments, a method of treating radiation dermatitis, comprising the administration of the topical composition to the skin of a subject is provided. In various embodiments, a method of preparing the topical composition is provided.

As used herein, the term “topical application” refers to the application or spread of a composition onto the surface of keratinous tissue, including, for example, human skin. As used herein, the terms “topical composition,” “topical skin care composition,” and “topical skin composition” are used interchangeably and include compositions suitable for topical application on keratinous tissue. In some embodiments, the topical compositions are dermatologically acceptable in that they do not have undue toxicity, incompatibility, instability, allergic response, and the like, when applied to the skin. In some embodiments, the topical compositions have a predetermined viscosity to avoid significant dripping or pooling after application to the skin.

As used herein, the term “skin” refers to the skin of all or one or more parts of the subject's body, particularly a human body, including the face, scalp, neck, chest, back, stomach, torso, legs, feet, underarms, hands, thighs, shins, arms, labial mucosa; and particularly a canine body, including the face, nose, back, neck, torso, stomach, tail, and legs.

In various embodiments, the topical composition comprises one or more herbal organic components. As used herein, an herbal component may include matter derived from a plant or plant part and used for consumption, including, for example, for medicinal, therapeutic, aromatic, and/or culinary purposes. In some embodiments, the matter derived from a plant can be obtained from one or more parts of the plant, including, for example, the whole fruit, whole vegetable, whole plant, whole tree, whole bush, seed, peel, fruit, stem, bark, leaf, root, flower, petal, bulb, etc.

Extracts from one or more species of the one or more herbal components can be obtained by extraction methods known to those of ordinary skill in the art. In some embodiments, the extract can be obtained from one or more portions of the plant, including, for example, the whole fruit, whole vegetable, whole plant, whole tree, whole bush, seed, peel, fruit, stem, bark, leaf, root, flower, petal, bulb, etc.

In some embodiments, the topical composition comprises the parts, extracts, or isolated compounds (alone or collectively, “herbal components”) from one or more plants, two or more plants, or a plurality of plants, selected from Japanese knotweed (Polygonum cuspidatum), Cortex Dictamni, Rhizoma Coptidis, Radix Sophorae Flavescentis (Sophora flavescens), Cortex Phellodendri, Calendula officinalis, Plantago lanceolata, Radix Arnebiae (also referred to as Radix Lithospermi), and Flos Carthami (also referred to as (Carthamus tinctorius) (safflower).

In various embodiments, the topical composition comprises one or more animal organic components. In some embodiments, the animal component is an invertebrate animal component, including, for example, an arthropod. In some embodiments, the topical composition comprises one or more centipede species, including, for example, one or more species from the genus Scolopendra. The animal component can be included in any suitable form. For example, in some embodiments, the animal component is a dried carcass.

In some embodiments, the topical composition is effective for treating one or more skin conditions on the skin of a subject, including radiation dermatitis. The listed herbal and animal components are known to have minimal or no side effects on human subjects.

In some embodiments, the topical composition comprises Japanese knotweed and one or more additional organic components selected from Cortex Dictamni, Rhizoma Coptidis, Radix Sophorae Flavescentis, Cortex Phellodendri, Calendula officinalis, Plantago lanceolata,Radix Arnebiae, Flos Carthami, and Scolopendra.

In some embodiments, the topical composition comprises Cortex Dictamni and one or more additional organic components selected from Japanese knotweed, Rhizoma Coptidis, Radix Sophorae Flavescentis, Cortex Phellodendri, Calendula officinalis, Plantago lanceolata,Radix Arnebiae, Flos Carthami, and Scolopendra.

In some embodiments, the topical composition comprises Rhizoma Coptidis and one or more additional organic components selected from Japanese knotweed, Cortex Dictamni,Radix Sophorae Flavescentis, Cortex Phellodendri, Calendula officinalis, Plantago lanceolata, Radix Arnebiae, Flos Carthami, and Scolopendra.

In some embodiments, the topical composition comprises Radix Sophorae Flavescentis and one or more additional organic components selected from Japanese knotweed, Cortex Dictamni, Rhizoma Coptidis, Cortex Phellodendri, Calendula officinalis, Plantago lanceolata, Radix Arnebiae, Flos Carthami, and Scolopendra.

In some embodiments, the topical composition comprises Cortex Phellodendri and one or more additional organic components selected from Japanese knotweed, Cortex Dictamni, Rhizoma Coptidis, Radix Sophorae Flavescentis, Calendula officinalis, Plantago lanceolata, Radix Arnebiae, Flos Carthami, and Scolopendra.

In some embodiments, the topical composition comprises Calendula officinalisand one or more additional organic components selected from Japanese knotweed, Cortex Dictamni, Rhizoma Coptidis, Radix Sophorae Flavescentis, Cortex Phellodendri, Plantago lanceolata, Radix Arnebiae, Flos Carthami, and Scolopendra.

In some embodiments, the topical composition comprises Plantago lanceolataand one or more additional organic components selected from Japanese knotweed, Cortex Dictamni, Rhizoma Coptidis, Radix Sophorae Flavescentis, Cortex Phellodendri, Calendula officinalis, Radix Arnebiae, Flos Carthami, and Scolopendra.

In some embodiments, the topical composition comprises Radix Arnebiae and one or more additional organic components selected from Japanese knotweed, Cortex Dictamni, Rhizoma Coptidis, Radix Sophorae Flavescentis, Cortex Phellodendri, Calendula officinalis, Plantago lanceolata, Flos Carthami, and Scolopendra.

In some embodiments, the topical composition comprises Flos Carthami and one or more additional organic components selected from Japanese knotweed, Cortex Dictamni, Rhizoma Coptidis, Radix Sophorae Flavescentis, Cortex Phellodendri, Calendula officinalis, Plantago lanceolata, Radix Arnebiae, and Scolopendra.

In some embodiments, the topical composition comprises Scolopendra and one or more additional organic components selected from Japanese knotweed, Cortex Dictamni, Rhizoma Coptidis, Radix Sophorae Flavescentis, Cortex Phellodendri, Calendula officinalis, Plantago lanceolata, Radix Arnebiae, and Flos Carthami.

In some embodiments, the one or more organic components were combined in equal parts based on weight to obtain an organic mixture. The organic mixture can be included in the topical composition in any suitable weight amount. In some embodiments, the topical composition is provided to a user in a container useful for dispensing the composition, such as, for example, a 2-ounce jar containing 50 grams of composition or a 4-ounce jar containing 100 grams of the composition. In some embodiments, the topical composition includes the organic mixture in an amount ranging from about 0.25% to about 25%, or about 0.5% to about 15%, or about 1% to about 10%, or about 1% to about 8%, or about 2% to about 6%, or about 3% to about 5% (by weight). In some embodiments, each of the respective organic components are provided in the organic mixture in a range of about 1 mg to about 1000 mg, or about 10 mg to about 750 mg, or about 100mg to about 500 mg, or about 300 mg to about 500 mg, or about 10 mg to about 250 mg, or about 15 mg to about 100 mg, etc., the amount for each being based on weight. For example, if the topical composition includes the organic mixture in an amount of about 5%, such as 5 grams in a 100-gram topical composition, and the organic mixture is composed of 10 specific organic components, then each of the ten organic components is provided in an amount of about 0.5 grams (500 mg).

In various embodiments, the one or more organic components described herein may be combined in any suitable concentration ratio when forming the topical composition. In some embodiments, for example, the topical composition comprises a plurality of herbal components. In some embodiments, the topical composition comprises the plurality of herbal components and one or more animal components. In some embodiments, each organic component is present in an equal amount (e.g., by weight), or a ratio of 1.0:1.0 respectively. In some embodiments, the respective organic components are present in different amounts. In some embodiments, two organic components are present at a ratio in the range of 0.10 to 10.0; 0.2 to 5.0; 0.5 to 2.5; or any range derivable therein (first organic component: second organic component). In some embodiments, two organic components are present at a ratio of 0.25:1.0; 0.5:1.0; 0.75:1.0; 1.0:1.0; 1.25:1.0; 1.50:1.0; 1.75:1.0; 2.0:1.0; etc. (first organic component: second organic component). In some embodiments, three or more organic components are present, and each respective component is present in an amount relative to one or each of the other components. For example, if the topical composition includes three organic components, the amount of the third component present may be expressed relative to the first component (1.0:1.0; third component: first component), the second component (1.0:0.5; third component: second component), or the first and second components (1.0:1.0:0.5; third component: first component: second component). In various embodiments, four or more, five or more, six or more, seven or more, eight or more, nine or more, ten or more, etc., organic components are present, and each respective organic component is present in an amount relative to one or each of the other components as described above.

In various embodiments, the topical composition comprises any number of combinations of the herbal and/or animal organic components. The concentrations of any organic components in the topical compositions can vary. In some embodiments, each of the respective organic components are provided in a range of from about 0.0001% to about 10%, or from about 0.001% to about 8%, or from about 0.01% to about 5%, or from about 0.1% to about 1%. In some embodiments, for example, the total amount for each respective organic component in the topical composition is about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.005 5%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.825%, 0.8500%, 0.8750%, 0.9000%, 0.9250%, 0.9500%, 0.9750%, 1.0%, 1.5%, 2.0%, 2.5%, 3.0%, 3.5%, 4.0%, 4. 5%, 5%, 5.5%, 6.0%, 6.5%, 7.0%, 7.5%, 8.0%, 8. 5%, 9.0%, 9.5%, or 10%; or any range derivable therein. The percentage can be calculated by weight or volume of the total composition. In some embodiments, the percentage is calculated by weight of the total topical composition. In some embodiments, the percentage is calculated by weight of the organic mixture portion of the topical composition. In some embodiments, the concentrations vary depending on the addition, substitution, and/or subtraction of one or more organic components in a respective composition.

In some embodiments, the one or more herbal components may include one or more flavonoids, terpenes, terpenoids, amino acids, proteins, sugars, enzymes, fatty acids, ester, and/or other organic compounds that are at least partially responsible for the beneficial activity of the topical composition. In some embodiments, the topical composition may include one or more additional (i.e., from a secondary source) flavonoids, terpenes, terpenoids, amino acids, proteins, sugars, enzymes, fatty acids, ester, and/or other organic compounds.

The one or more herbal components of the topical composition may be included in any suitable form. In some embodiments, for example, the one or more herbal components are processed to form extracts, dried, powdered, pelleted, concentrated, gelled, etc. In some embodiments, the one or more herbal components are dried and powdered, and then incorporated into the topical composition. In some embodiments, for example, the one or more herbal components are maintained as an aqueous extract, alcohol extract, or a combination of aqueous/alcohol extract. In some embodiments, one or more herbal components of the topical composition can be extracted with a suitable solvent (e.g., ethanol), combined with one or more additional herbal components, and concentrated to a concentration suitable for the topical composition. In some embodiments, the resulting concentrate of the topical composition is dried to provide a powdery composition, which may be useful for dosing or storage of the herbal components. In some embodiments, the one or more herbal components are formulated for nebulization, vaporization, or another method of inhalation.

In some embodiments, the topical composition has a pH of 1, 2, 3, 4, 5, 6, 7, 8, 9, 10, 11, 12, 13, or 14. In some embodiments, the topical composition has a pH in the range of about 4 to about 8, or in the range of about 5 to about 7. In some embodiments, the topical composition has a pH in the range of about 5.5 to about 6.

In some embodiments, the viscosity of the topical composition is selected to achieve a desired result (e.g., depending on the type of composition desired, the viscosity of such composition can be from about 1 cps to well over 1 million cps or any range or integer derivable therein (e.g., 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000 cps, etc., as measured on a Brookfield Viscometer using a TC spindle at 2.5 rpm at 25° C.). In some embodiments, the topical composition has a predetermined viscosity to avoid significant dripping or pooling after application to the skin.

The dose of the topical composition is not particularly limited and may be appropriately selected depending on the condition or body weight of the patient, the severity of disease or condition, the specific formulation, and/or the route, period, and/or schedule of administration. For example, the topical composition may be administered in an amount ranging from 0.0001 to 1000 g/kg per day on a dry weight basis, or 0.001 to 100 g/kg per day, 0.001 to 10 g/kg per day, 0.001 to 1 g/kg per day, 0.0001 g/kg or more per day, 0.001 g/kg or more per day, 0.05 g/kg or more per day, 0.01 g/kg or more per day, or 0.005 g/kg or more per day.

In some embodiments, the topical composition is formulated for topical skin application one or more times per day. For example, in some embodiments, the topical composition is formulated for an administration schedule of twice per day, three times per day, four times a day, etc. In some embodiments, the topical composition is formulated for long-term storage (e.g., 3 or more years). In some embodiments, the topical composition is storage stable and/or color stable.

The topical composition may be adapted for administration by any appropriate route. In some embodiments, the topical composition may be delivered through a surface of the subject's body, e.g., transdermally or transmucosally. As used herein, “transdermal” means passage into and through the skin to achieve effective therapeutic blood levels. As used herein, “transmucosal” means passage through a mucosal membrane of a living organism, and therefore includes delivery of through sublingual, oral patch, or buccal tissue. Transdermal or transmucosal delivery will involve topical application of the topical composition in the form of an ointment, salve, lotion, serum, emulsion, gel, cream, powder, spray, aerosol, nebulizer solution, or the like, or may involve use of a drug delivery device.

In some embodiments, the topical composition comprises one or more carriers, excipients, diluents, stabilizers, lubricants, moisturizers, and/or solubilizers, such as those used for various routes of topical administration. In such embodiments, diluents and excipients, such as fillers, extenders, binders, thickening agents, emulsifying agents, structuring agents, wetting agents, lubricants, disintegrants, or surfactants, may be used. In some embodiments, the topical composition comprises one or more pharmaceutical agents, silicone compounds, antioxidants, essential oils, botanical extracts, cosmetic components, preservatives, probiotics, probiotic derivatives, or prebiotics. The topical compositions may optionally contain a permeation enhancer to increase the rate at which the herbal composition permeates through the skin or mucosal tissue.

### Carriers and Formulations

In some embodiments, the topical composition comprises a dermatologically acceptable carrier (also referred to as a vehicle). Example carriers include water, glycerin, ethanol, chlorphenesin, sodium metabisulfite, oil (e.g., olive oil, coconut oil), or a combination thereof.

In some embodiments, the topical composition can be incorporated into one or more types of carriers. Examples of suitable vehicles include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, and ointments or by other method or any combination of the forgoing as would be known to one of ordinary skill in the art. Variations and other appropriate vehicles would be apparent to one of ordinary skill in the art. In some embodiments, the concentrations and combinations of the compounds, components, and agents are selected such that the combinations are chemically compatible and do not form complexes that precipitate from the finished product.

In some embodiments, the topical composition can be used in any moisturizing creams, skin benefit creams and lotions, day lotions, gels, ointments, night creams. In some embodiments, the topical composition can be formulated as a leave-on or rinse-off product.

In some embodiments, the topical composition can be encapsulated for delivery to a target area such as skin. Examples of encapsulation techniques include the use of liposomes, vesicles, and/or nanoparticles (e.g., biodegradable and non-biodegradable colloidal particles comprising polymeric materials in which the component is trapped, encapsulated, and/or absorbed; including, for example nanospheres and nanocapsules) that can be used as delivery vehicles to deliver the topical composition to skin.

### Moisturizing Agents

In some embodiments, the topical composition comprises one or more moisturizing agents. Examples of moisturizing agents include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol. Further examples include acetylated lanolin, acetylated lanolin alcohol, alanine, algae extract, aloe barbadensis, aloe-barbadensis extract, aloe barbadensis gel, allantoin, althea officinalis extract, apricot (prunus armeniaca) kernel oil, arginine, arginine aspartate, arnica montana extract, aspartic acid, avocado (persea gratissima) oil, barrier sphingolipids, butyl alcohol, beeswax, behenyl alcohol, beta-sitosterol, birch (betula alba) bark extract, borage (borago officinalis) extract, butcherbroom (ruscus aculeatus) extract, butylene glycol, calendula officinalis extract, calendula officinalis oil, Calophyllum inophyllum (foraha) nut oil, candelilla (euphorbia cerifera) wax, canola oil, caprylic/capric triglyceride, cardamon (elettaria cardamomum) oil, carnauba (copernicia cerifera) wax, carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, clove leaf oil, cocoa (theobroma cacao) butter, coco-caprylate/caprate, coconut (cocos nucifera) oil, collagen, collagen amino acids, corn (zea mays) oil, fatty acids, decyl oleate, dimethicone copolyol, dimethiconol, dioctyl adipate, dioctyl succinate, dipentaerythrityl hexacaprylate/hexacaprate, DNA, emu oil, erythritol, ethoxydiglycol, ethyl linoleate, eucalyptus globulus oil, evening primrose (oenothera biennis) oil, fatty acids, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrognated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, maltitol, matricaria (chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, pomegranate seed oil (unica granatum), potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinol palmitate, rice (oryza sativa) bran oil, RNA, rosemary (rosmarinus officinalis) oil, rose oil, rosehip seed oil, safflower (carthamus tinctorius) oil, sage (salvia officinalis) oil, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tea tree oil (Malaleuca alternifolia), tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

### Antioxidants

In some embodiments, the topical composition comprises one or more antioxidant agents. Examples of antioxidants include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphit.

### Thickening Agents

In some embodiments, the topical composition comprises one or more thickening agents. As used herein, a thickening agent, or thickener, or gelling agent, includes one or more substances that can increase the viscosity of the composition. In some embodiments, thickeners increase the viscosity of the composition without substantially modifying the efficacy of the one or more active components within the composition. In some embodiments, thickeners increase the stability of the composition. In some embodiments, thickeners include hydrogenated polyisobutene or trihydroxystearin, or a mixture of both.

In various embodiments, thickening agents include carboxylic acid polymers, crosslinked polyacrylate polymers, polyacrylamide polymers, polysaccharides, and gums. Examples of crosslinked polyacrylate polymers include cationic and nonionic polymers.

Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Further examples of carboxylic acid polymers include carbomers, which are homopolymers of acrylic acid crosslinked with allyl ethers of sucrose or pentaerytritol.

Examples of polyacrylamide polymers (including nonionic polyacrylamide polymers having substituted, branched, or unbranched polymers) include polyacrylamide, isoparaffin and laureth-7, multi-block copolymers of acrylamides and substituted acrylamides with acrylic acids and substituted acrylic acids.

Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Further examples include an alkyl-substituted cellulose in which one or more of the hydroxy groups of the cellulose polymer is hydroxyalkylated (e.g., hydroxy ethylated or hydroxypropylated) to form a hydroxyalkylated cellulose that can then be modified with a C₁₀-C₃₀ straight chain or branched chain alkyl group through an ether linkage. In some embodiments, the polymers are ethers of C₁₀-C₃₀ straight or branched chain alcohols with hydroxyalkylcelluloses. Further examples of polysaccharides include scleroglucans comprising a linear chain of (1-3) linked glucose units with a (1-6) linked glucose every three unit.

Examples of gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

### Emulsifying Agents

In some embodiments, the topical composition comprises one or more emulsifiers. As used herein, an emulsifier is an agent that can reduce the interfacial tension between phases and improve the formulation and stability of an emulsion. The emulsifiers can be nonionic, cationic, anionic, and zwitterionic emulsifiers. Examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

### Surfactants

In some embodiments, the topical composition comprises a surfactant. As used herein, a surfactant is a compound that reduces surface tension between two liquids, or between a liquid and a solid. Surfactants can also function as cleaning agents, wetting agents, emulsifiers, foaming agents, and dispersants. Examples of surfactants include coco glucoside, ethoxylated fatty alcohol ethers, PEG castor oil, PEG esters, propylene glycol esters, glyceryl esters and derivatives, polymer ethers, sorbitan derivatives, and aliphatics. Examples include alcohols, emulsified waxes, and mixtures thereof. Exemplary ethoxylated aliphatic alcohol ethers are not particularly limited, but are limited, but are not limited to: steareth-2, steareth-10, steareth-20, steareth-21, steareth-40, steareth-100, behenes-10, ceteares-2, and ceteares.-3, Ceteares-5, Ceteares-6, Ceteares-10, Ceteares-12, Ceteares-15, Ceteares-20, Ceteares-21, Ceteares-22, Ceteares-25, Ceteares-30, Ceteares-31, Ceteares-32, Ceteares-33, Setes-2, Setes-10, Setes-20, Setes-23, Corres-24, Isocetes-20, Laureth-2, Laureth-3, Laureth-4, Laureth-5, Laureth-9, Laureth -10, Laureth-12, Laureth-15, Laureth-20, Laureth-21, Laureth-22, Laureth-23, Nonoxinol-9, Nonoxinol-15, Octoxinol-1, Octoxinol-9, Oles-2, Oles-5, Oles-10, Oles-20, C20-40 Palace-24 and Trideces-10, and mixtures thereof. Exemplary PEG castor oils include PEG-7 hydride castor oil, PEG-25 hydride castor oil, PEG-30 castor oil, PEG-33 castor oil, PEG-35 castor oil, PEG-36 castor oil, PEG-40 castor oil, PEG-40 hydride castor oil, PEG-50 castor oil, PEG-54 hydride castor oil, PEG-60 castor oil and PEG-60 hydride castor oil, and mixtures thereof. Can be mentioned. Exemplary PEG esters are not particularly limited, but are PEG-4 dilaurate, PEG-150 distearate, PEG-12 glyceryl laurate, PEG-120 glyceryl stearate, PEG-6 isostearate, PEG-4 laurate., PEG-8 laurate, PEG-20 methyl glucose sesquistearate, PEG-5 oleate, PEG-6 oleate, PEG-10 oleate, PEG-25 propylene glycol stearate, PEG-2 stearate, stearic acid PEG-6, PEG-6-32 stearate, PEG-8 stearate, PEG-9 stearate, PEG-20 stearate, PEG-40 stearate, PEG-45 stearate, PEG-50 stearate and stearic acid PEG-100, as well as mixtures thereof. Exemplary propylene glycol esters include propylene glycol laurate, propylene glycol palmitostearate, propylene glycol ricinoleic acid and propylene glycol stearate, and mixtures thereof. Exemplary glyceryl esters and derivatives include glyceryl behenate, glyceryl dibechenate, glyceryl dioleate, glyceryl distearate, glyceryl isostearate, glyceryl laurate, glyceryl linoleate, glyceryl monostearate, oleic acid. Glyceryl, glyceryl palmitate, glyceryl lysinolate, glyceryl stearate, PEG-23 glyceryl cocoate, PEG-6 caprylic acid/glyceryl caprate, PEG-7 glyceryl cocoate, polyglyceryl distearate- 10, polyglyceryl diisostearate-2, polyglyceryl hypostearate-3 and polyglyceryl diisostearate-6, PEG-12 glyceryl laurate, PEG-120 glyceryl stearate, and mixtures thereof. Exemplary polymer ethers include poloxamer 124, poloxamer 181, poloxamer 182, poloxamer 184, poloxamer 188, poloxamer 237, poloxamer 331, poloxamer 338 and poloxamer 407, and mixtures thereof. Exemplary sorbitan derivatives include polysorbate 20, polysorbate 40, polysorbate 60, polysorbate 65, polysorbate 80, sorbitan monolaurate, sorbitan monooleate, sorbitan monopalmitate, sorbitan monostearate, and sorbitan sesqui. Includes oleate, sorbitan trioleate and sorbitan tristearate, and mixtures thereof. Exemplary aliphatic alcohols include isostearyl alcohols, caprylyl alcohols, decyl alcohols, lauryl alcohols, myristyl alcohols, behenyl alcohols, lanolin alcohols, arachidyl alcohols, oleyl alcohols, palm alcohols, isosetyl alcohols, cetyl alcohols, stearyl alcohols, and cetearyl alcohols, and mixtures thereof. Exemplary emulsifying waxes include a mixture of cetearyl alcohol and polysorbate 60.

### Structuring Agents

In some embodiments, the topical composition comprises a structuring agent. As used herein, a structuring agent modifies the rheological characteristics of the composition to contribute to the composition's stability. In some embodiments, the structuring agent also functions as an emulsifier or surfactant. Examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

### Preservatives

In some embodiments, the topical composition comprises one or more preservatives. Examples of preservatives include quaternary ammonium preservatives, such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal, or combinations thereof.

### Silicone Compounds

In some embodiments, the topical composition comprises one or more silicone containing compounds. As used herein, a silicone containing compound includes any member of a family of polymeric products having a molecular backbone that is made up of alternating silicon and oxygen atoms with side groups attached to the silicon atoms. In such embodiments, the-Si—O-chain lengths, side groups, and crosslinking can be varied. As such, the silicone containing compounds can be synthesized into a wide variety of materials. They can vary in consistency from a liquid to gel form. Examples of silicone containing compounds include silicone oils (e.g., volatile and non-volatile oils), gels, and solids. In some embodiments, the silicon containing compounds includes a silicone oil such as a polyorganosiloxane. Examples of polyorganosiloxanes include dimethicone, cyclomethicone, polysilicone-11, phenyl trimethicone, trimethylsilylamodimethicone, stearoxytrimethylsilane, or mixtures of these and/or other organosiloxane materials in any given ratio to achieve the desired consistency and application characteristics depending upon the intended application (e.g., to a particular area such as the skin, hair, or eyes). As used herein, a “volatile silicone oil” includes a silicone oil having a low heat of vaporization, i.e., normally less than about 50 cal per gram of silicone oil. Examples of volatile silicone oils include cyclomethicones; low viscosity dimethicones, i.e., dimethicones having a viscosity of about 50 cst or less (e.g., dimethicones).

### Essential Oils

In some embodiments, the topical composition comprises one or more essential oils. Essential oils include oils derived from herbs, flowers, trees, and other plants. Such oils can be present as tiny droplets between the plant's cells and can be extracted by methods known to those of skill in the art (e.g., steam distilled, enfleurage (i.e., extraction by using fat), maceration, solvent extraction, or mechanical pressing). When essential oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160 to 240° C. and densities ranging from about 0.759 to about 1.096.

Essential oils are generally named by the plant from which the oil is found. For example, rose oil or peppermint oil are derived from rose or peppermint plants, respectively. Examples of essential oils include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

### Botanical Extracts

In some embodiments, the topical composition comprises one or more botanical extracts. Botanical extracts are derived from various parts of herbs, trees, and other plants including, the flowers, stems, seeds, roots, and fruit thereof. In some embodiments, the extracts include one or more of the following: absinthium, aconite, agar, algin, allantoin, aloe vera, althea root, ambrette seed, angelica, anise, Arnica Montana, asafetida, astragalus, avocado, lemon balm, barberry, basil sweet, bamboo (Bambusa vulgaris), bayberry bark, bee pollen, beeswax, benzoin, bilberry, bisabolol, blood root, Boswelia serrata, borage, buckthorn, buckwheat honey, burdock, cade oil, calamus, calendula, cananga oil, caraway, cardamon, cascara sagrada, cascarilla bark, celery seed, chamomile, chaparral, chenpodium, chicory root, coatis (Rhizoma coptidis), comfrey root, cortex phellodendri chinensis (amur cork tree bark) dandelion root, dictamnus root bark (cortex dictamni) echinacea, eleuthero, eucalyptus, evening primrose, fangfeng, fenugreek, feverfew, Fo-ti, ganoderma, genet, geranium (wild), ginko, ginseng, gotu kola, giant knotweed (rhizoma polygonie cuspidate) rhizome honey, hops, horse chestnut, horsetail, hypericum, Irish moss, job's tears, jojoba, jujube, licorice root, lycium fruit, magnolia flower, melatonin, neem, parsley, pisum sativum (pea) seed extract, poria, quillaia, radix sophorea flavescetis (light yellow spoor root), rehmannia, rhoiola rosea root, rose hips, rosemary, rubarb root (rhizoma rhei), sage, sarsaparilla, sea kelp, tannic acid, thyme, willow bark, witch hazel, yarrow, and yucca.

### Additional Herbal Extracts

In some embodiments, the topical composition comprises one or more additional plant extracts. In some embodiments, the extracts are from plants used in traditional Chinese medicine for their healing, anti-swelling, wound healing, anti-inflammatory, or antihistaminic properties. In some embodiments, the herbs can include: atractylodes (baizhu and cangzhu), baizhu (atractylodes), bletilla tuber (baiji), cangzhu (atractylodes), dahurian angelica (baizhi), dittany bark (baixianpi), forsythia fruit (lianqiao), garden burnet (diyu), honeysuckle flower (jinyinhua), knotweed, giant (huzhang), ligusticum (gaoben), luffa (sigualuo), mume (smoked plum or wumei), pearl (zhenzhu or margarita), mother of pearl (zhenzhumu), peony bark and peony root (mudapi, shaoyao: chishaoyao and baishoyao), phellodendron bark (hungbai, purslane, common, machixian), red sage (danshen), safflower (false saffron: honghua), sichuan lovage (chuanxiog), skullcap, and baikal (huangqin).

### Pharmaceutical Agents

In some embodiments, the topical composition comprises one or more pharmaceutical agents. Examples of pharmaceutical agents include existing agents used to treat dermatitis, analgesics, anesthetics, antihistamines, anti-inflammatory agents including non-steroidal anti-inflammatory drugs, antibiotics, antifungals, antivirals, antimicrobials, antipruritics, antipsoriatic agents, antiseborrheic agents, biologically active proteins and peptides, burn treatment agents, diaper rash treatment agents, skin protectant and/or barrier agents, corticosteroids (e.g., hydrocortisone), sunburn treatment agents, sunscreens, wound treatment agents, wound healing agents, and cannabinoids that mitigate itch sensation and inflammation.

### Probiotics, Probiotic Derivatives, and Prebiotics

In some embodiments, the topical composition comprises one or more probiotics, probiotic derivatives, or prebiotics. In some embodiments, the probiotics, probiotic derivatives, or prebiotics are administered daily, including, for example, once per day, twice daily, three times per daily, etc. In some embodiments, the one or more probiotic strains can be selected from Lactobacillus brevis, Lactobacillus johnsonnii, Lactobacillus salibarius, Lactobacillus paracasei, Stretococcus thermophilus, Bifidobacterium longum, Roseomonas mucosa, Vitresoscilla filiformis, and kefir gel. In some embodiments, the prebiotic is avena sativa (oat) kernel extract. In some embodiments, the one or more probiotics, probiotic derivatives, or prebiotics are formulated or administered with one or more compounds, including acetic acid, diacetyl (butane-2,3-dione), sphingomyelinase, hyaluronic acid, lipoteichoic acid and peptidoglycan, and lactic acid.

### Cosmetic Components

In some embodiments, the topical composition comprises one or more cosmetic components. Examples include fragrances (artificial and natural), dyes and color components (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraminobenzoic acid (PABA) and corresponding PABA derivatives, titanium dioxide, zinc oxide, etc.), essential oils, vitamins (e.g. A, B, C, D, E, and K), trace metals (e.g., zinc, calcium and selenium), anti-irritants (e.g. steroids and non-steroidal anti-inflammatories), botanical extracts (e.g., chamomile, cucumber extract, ginkgo biloba, ginseng, and rosemary), anti- microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), humectants (e.g., sorbitol, urea, and mannitol), magnesium and/or aluminum hydroxide stearate, and skin conditioning agents (e.g., aloe extracts, allantoin, bisabolol, dimethicone, hyaluronic acid, and dipotassium glycyrrhizate).

In some embodiments, the composition comprises, in final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99%, or any range derivable therein, of at least one of the components mentioned throughout this disclosure. The percentage can be calculated by weight or volume of the total composition. In some embodiments, the concentrations vary depending on the addition, substitution, and/or subtraction of one or more components in a respective composition.

The various ingredients above are listed in specific categories (e.g., carrier, moisturizer, structuring agent, etc.) for convenience only. In some embodiments, a particular ingredient may be included in the topical composition for multiple reasons or functions. For example, the topical composition may include a particular ingredient for its properties and ability to function as a carrier and as a moisturizer. Any ingredient listed under one specific category may be used for an alternative purpose, and no unnecessary limitations should be read into the claims based on the specific categories.

### EXAMPLES

The following examples are provided to aid in the understanding of the present disclosure, the true scope of which is set forth in the appended claims. One of skill in the art would appreciate that modifications can be made in the compositions and/or procedures set forth without departing from the spirit of the disclosure.

### Method of Preparing Topical Compositions

Example 1. The following organic components were used in this example: Japanese knotweed (Polygonum cuspidatum), Cortex Dictamni, Rhizoma Coptidis, Radix Sophorae Flavescentis (Sophora flavescens), Cortex Phellodendri, Calendula officinalis, Plantago lanceolata, Radix Arnebiae, Flos Carthami, and Scolopendra.

The organic components were combined in equal parts based on weight and ground using a ball-mill grinder until an average particle size of 500 micrometers was achieved. For example, a 100-gram composition would comprise 10 grams, by weight, of each of the ten organic components. The ground composition was then cooled to zero degrees Celsius (0° C.).

Next, a cold ethanol extraction method was used to minimize lipid and chlorophyl contamination of the final product. The solvent (ethanol) was cooled to negative 40 degrees Celsius (−40° C.) and then combined with the pre-cooled ground composition at a ratio of 1:4 (organic composition to ethanol).

This maceration was placed in a sealed reaction vessel and left for 12 hours at negative 30 degrees Celsius (−30° C.). The maceration was then passed through a 200-micron filter and transferred into the evaporation flask configured for a rotary evaporator. The solvent was then removed using the rotary evaporator and the resulting residue (the “organic composition residue”) was collected from the evaporation flask.

Lipid Phase (55% of 100 grams). Beeswax (17.5 grams, 17.50% weight by weight (w/w)), coconut oil (27.5 grams, 27.50% w/w), and stearic acid (10.0 grams, 10.00% w/w) were weighed and added to a reaction vessel, which was then heated to 70 degrees Celsius (70° C.) under constant stirring until all components have fully melted.

Water-miscible phase (45% of 100 grams). The organic composition residue (100 grams, 4.38%), coco glucoside (5 grams, 5.00%), glycerin (22.50 grams, 22.50%), deionized water (6.56%), and ethanol (6.56%) were combined in a second reaction vessel and heated to 70 degrees Celsius (70° C.) under continuous stirring. All relative concentration amounts are expressed in weight concentration with regard to the final combination (100 grams) before the sonication discussed below.

The lipid phase solution (55 grams) and the water-miscible phase (45 grams) solution were transferred from their respective reaction vessels to a third vessel heated at 60 degrees Celsius (60° C.), into which a 500 W, 20 KHz ultrasonic processing probe was introduced to create nanoscopic liposomes (“nano-emulsion”) via the shear stress generated from the ubiquitous cavitations induced by the probe. Ultrasonication was continued under these conditions for 10 minutes. The vessel and resulting product were then placed in a water bath that gradually cools for 30 minutes from 60° C. to 40° C., with constant stirring in the vessel. The vessel was then allowed to rest at room temperature (about 20 degrees Celsius (20° C.)) for 24 hours.

Example 2. The following organic components were used in this example: Japanese knotweed (Polygonum cuspidatum), Cortex Dictamni, Rhizoma Coptidis, Radix Sophorae Flavescentis (Sophora flavescens), Cortex Phellodendri, Calendula officinalis, Plantago lanceolata, Radix Arnebiae, Flos Carthami, and Scolopendra.

The organic components were combined in equal parts based on weight and ground using a ball-mill grinder until an average particle size of 500 micrometers was achieved. For example, a 100-gram composition would comprise 10 grams, by weight, of each of the ten organic components. The ground mixture was then combined with glycerol at room temperature at a ratio of 1:5 (organic composition to glycerol). For example, 100 grams of the ground organic mixture would be combined with 500 grams of glycerol.

The maceration (glycerol and organic mixture) was added to a reaction vessel and placed in a water bath at 90 degrees Celsius (90° C.) for 12 hours. The mixture was then cooled to room temperature for 48 hours, and then passed through a 600-micron filter (e.g., stainless steel filter). The resultant solution was used to create a nano-emulsion.

Lipid Phase (75.75 grams). Beeswax (30.3 grams, 30.3% weight by weight (w/w)), coconut oil (33.33 grams, 33% w/w), and stearic acid (12.12 grams, 12.12% w/w) were weighed and added to a first reaction vessel, and then heated to 70 degrees Celsius (70° C.) under constant stirring until all components are fully melted.

Water-miscible phase (24.24 grams). The organic composition solution (12.12 grams, 12.12% weight by weight (w/w)) and coco glucoside (12.12 grams, 12.12%) were combined in a second reaction vessel and heated to 70 degrees Celsius (70° C.) under continuous stirring. All relative concentration amounts are expressed in weight concentration with regard to the final combination (100 grams).

The solutions of the first and second reactions vessels were then transferred to a third reaction vessel, heated to 60 degrees Celsius (60° C.), into which a 500 W, 20 KHz ultrasonic processing probe was introduced to create nanoscopic liposomes (“nano-emulsion”) via the shear stress generated from the ubiquitous cavitations induced by the probe. Ultrasonication was continued for 10 minutes. The reaction vessel was then placed in a water bath for 30 minutes while gradually reducing the cooling the temperature from 60° C. to 40° C., with constant stirring. The vessel was then allowed to rest at room temperature for 24 hours.

### Method of Administering Topical Composition to a Subject

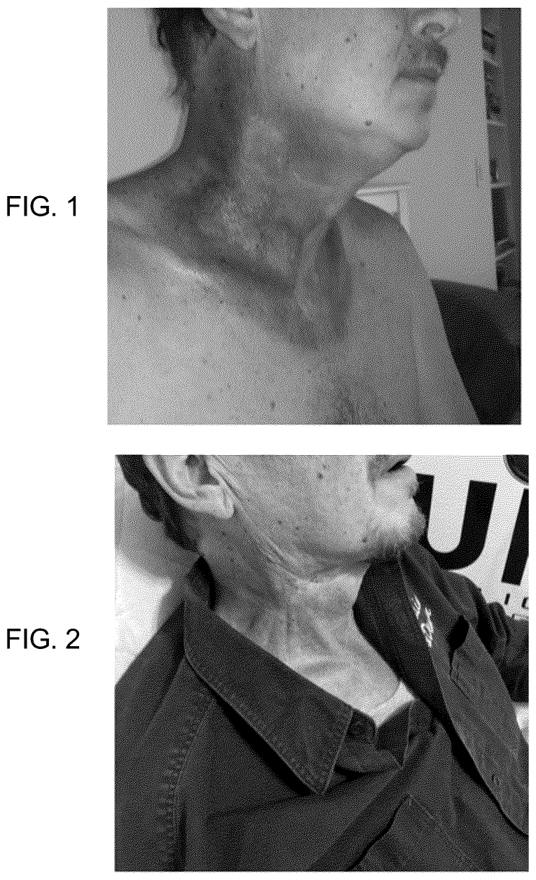

A topical composition comprising the nano-emulsion topical composition prepared according to the method above was administered to a subject in need thereof. Details pertaining to the subject, the treatment, and the results are provided in the table below.

| No. | Subject Description and Results | Outcome |
|---|---|---|
| 1 | A 67-year-old male human subject having a history of 48 topical radiation | + |
| | treatments for diagnosed head and neck cancer. The subject had severe | |
| | radiation burns of his head and neck after radiation oncology | |
| | treatments. After two and a half weeks of twice daily treatment with the | |
| | herbal salve of Example 1, the radiation burn was ameliorated. Before and | |
| | after images of the subject are provided in FIGS. 1 and 2. | |
| 2 | A 42-year-old female human subject having a triple negative breast cancer | + |
| | of the right breast diagnosis. The subject had a history of six radiation | |
| | treatments for breast cancer, which left her with redness, skin blistering, | |
| | and dryness. The subject was treated with the herbal salve of Example 1, | |
| | twice daily, for two weeks and the radiation burn was ameliorated. | |
| 3 | An 8-year-old male canine subject diagnosed with a thyroid tumor was | + |
| | given 4 radiation treatments per week for a few weeks. The dog owners | |
| | applied the herbal salve immediately after each radiation treatment. The | |
| | organic herbal salve of Example 1 prevented any radiation burns from | |
| | forming due to prophylactic application. | |
| 4 | A 12-year-old female canine subject was diagnosed with Basal Cell | + |
| | Carcinoma of the nose, which produced significant scarring, blistering, | |
| | redness, and damage to the nasal tissue. The dog owners applied the | |
| | herbal salve of Example 1 topically daily for one month to ameliorate the | |
| | damage to the dog's nose. | |

Legend: + indicates a positive subject outcome, with no reported negative side effects.

In some embodiments, the method comprises topically applying the topical composition in an amount effective to reduce or ameliorate any damage to the skin. In some embodiments, the method comprises topically applying the topical composition to the subject in an amount effective to prevent damage to skin or to mitigate the risk of damage to the skin.

Exemplary embodiments of the topical compositions and methods are described above in detail. The topical compositions and methods are not limited to the specific embodiments described herein, but rather, components of the topical compositions and/or steps of the method may be utilized independently and separately from other components and/or steps described herein. It is contemplated that any embodiment discussed in this specification can be implemented with respect to any method or composition of the disclosure, and vice versa. Furthermore, compositions of the disclosure can be used to achieve methods of the disclosure.

The recitation of a range of values herein is intended to serve as a shorthand for referring individually to each separate value falling within the range, including the endpoints, unless otherwise indicated herein, and each separate value is incorporated into the specification as if it were individually recited herein. For example, a range of about 5% to about 10% includes 5%, 6%, 7%, 8%, 9%, and 10%, as well as any subranges, including about 5% to about 7%.

All methods described herein can be performed in any suitable order unless otherwise indicated herein or otherwise clearly contradicted by context. As will be understood by one skilled in the art, ranges disclosed herein encompass any and all possible subranges and combinations of subranges thereof. Any listed range can be easily recognized as sufficiently describing the same range being broken down into at least equal halves, thirds, quarters, fifths, tenths, etc. As a non-limiting example, each range discussed herein can be readily broken down into a lower third, middle third and upper third, etc. As will also be understood by one skilled in the art, language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a composition having 1-3 components refers to compositions having 1, 2, or 3 components. Similarly, a composition having 1-5 components refers to compositions having 1, 2, 3, 4, or 5 components, and so forth.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, the use of examples, or exemplary language (e.g., “such as”), is intended to illuminate the embodiments and does not pose a limitation on the scope of the claims unless otherwise stated. No language in the specification should be construed as indicating any non-claimed element as essential.

As used herein, the terms “about” and “substantially” will be understood by persons of ordinary skill in the art and will vary to some extent depending upon the context in which it is used. If there are uses of the term which are not clear to persons of ordinary skill in the art, given the context in which it is used, “about” and “substantially” will mean up to plus or minus 10% of the particular term.

As used herein, the terms “subject,” “individual,” or “patient” can be an individual organism, a vertebrate, a mammal, or a human. “Mammal” includes a human, non- human primate, murine (e.g., mouse, rat, guinea pig, hamster), ovine, bovine, ruminant, lagomorph, porcine, caprine, equine, canine, feline, avis, etc. In some embodiments herein, the mammal is human. In some embodiments herein, the mammal is canine.

The term “administering” a composition to a subject means delivering the composition to the subject. “Administering” can include prophylactic administration of the composition (i.e., before one or more symptoms of a skin disease or condition are detectable) and/or therapeutic administration of the composition (i.e., after the skin disease or condition and/or one or more symptoms of the skin disease or condition are detectable). The methods of some embodiments may include administering one or more compounds, compositions, or agents. If more than one compound, composition, or agent is to be administered it may be administered together at substantially the same time, and/or be administered before, concomitantly with, and/or after administration of another composition or therapeutic procedure (e.g., acupuncture).

As used herein, the terms “effective amount” or “therapeutically effective amount,” refer to a quantity sufficient to achieve a desired therapeutic and/or prophylactic effect, e.g., an amount which results in the full or partial amelioration of disorders or symptoms, e.g., those associated with a skin disease or condition in a subject in need thereof. In the context of therapeutic or prophylactic applications, the amount of a composition administered to the subject will depend on the type and severity of the disorder or symptom and on the characteristics of the individual, such as general health, age, sex, body weight, and tolerance to drugs or herbs. It will also depend on the degree, severity, and type of disorder or symptom. The compositions can also be administered in combination with one or more additional compounds, compositions, or herbs. In some embodiments, multiple doses are administered. In some embodiments, multiple therapeutic compositions or compounds are administered. In the methods described herein, the compositions may be administered to a subject having one or more signs or symptoms of a disorder described herein.

This written description uses examples to disclose the present embodiments, including the best mode, and also to enable any person skilled in the art to practice the present embodiments, including making and using any herbal compositions or performing any methods. The patentable scope of the present embodiments is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

I/We claim:

1. A composition, comprising two or more organic components selected from the group consisting of:
Japanese knotweed,
Cortex Dictamni,
Rhizoma Coptidis,
Radix Sophorae Flavescentis,
Cortex Phellodendri,
Calendula officinalis,
Plantago lanceolata,
Radix Arnebiae,
Flos Carthami, and
Scolopendra;
wherein each respective organic component of the two or more organic components is included in an amount ranging from about 0.01% to about 5% w/w.

2. The composition of claim 1, wherein each of the two or more organic components is included in an amount ranging from about 0.1% to about 1% w/w.

3. The composition of claim 1, comprising each of the respective organic components.

4. The composition of claim 3, further comprising coco glucoside, glycerin, water, and ethanol.

5. The composition of claim 4, comprising:
about 3% to about 6% of the respective organic components;
about 3% to about 6% of the coco glucoside;
about 20% to about 25% of the glycerin;
about 4% to about 8% of the water; and
about 4% to about 8% of the ethanol;
wherein the respective amounts are based on weight.

6. The composition of claim 1, further comprising coco glucoside and glycerin.

7. The composition of claim 6, further comprising beeswax, coconut oil, and stearic acid.

8. A topical composition for treating radiation dermatitis, comprising a plurality of organic components selected from:
Japanese knotweed,
Cortex Dictamni,
Rhizoma Coptidis,
Radix Sophorae Flavescentis,
Cortex Phellodendri,
Calendula officinalis,
Plantago lanceolata,
Radix Arnebiae,
Flos Carthami,
Scolopendra;
wherein each of the two or more organic components is included in an amount ranging
from about 0.01% to about 5%.

9. The topical composition of claim 8, further comprising coco glucoside and glycerin.

10. The topical composition of claim 8, comprising:
about 3% to about 6% of the organic components;
about 3% to about 6% of coco glucoside;
about 20% to about 25% of glycerin;
about 4% to about 8% of water; and
about 4% to about 8% of ethanol;
wherein the respective amounts are based on weight.

11. The topical composition of claim 8, further comprising beeswax, coconut oil, and stearic acid.

12. A method of treating a skin condition in a subject in need thereof, comprising:
administering a topical composition to the subject, the topical composition comprising two or more organic components selected from the following:
Japanese knotweed,
Cortex Dictamni,
Rhizoma Coptidis,
Radix Sophorae Flavescentis,
Cortex Phellodendri,
Calendula officinalis,
Plantago lanceolata,
Radix Arnebiae,
Flos Carthami, and Scolopendra;
wherein each of the two or more organic components is included in an amount ranging from about 0.01% to about 5%.

13. The method of claim 12, wherein the skin condition is dermatitis or a burn.

14. The method of claim 13, wherein the dermatitis is radiation dermatitis and wherein the burn is a radiation burn.

15. The method of claim 12, wherein the topical composition comprises each of the respective organic components.

16. The method of claim 12, wherein each of the two or more organic components is included in an amount ranging from about 0.1% to about 1% w/w.

17. The method of claim 12, further comprising coco glucoside and glycerin.

18. The method of claim 12, wherein the topical composition comprises:
about 3% to about 6% of the respective organic components;
about 3% to about 6% of coco glucoside;
about 20% to about 25% of glycerin;
about 4% to about 8% of water; and
about 4% to about 8% of ethanol;
wherein the respective amounts are based on weight.

19. The method of claim 17, wherein the topical composition further comprises beeswax, coconut oil, and stearic acid.

20. The method of claim 17, wherein the topical composition comprises:
4% to 5% of the respective organic components;
4% to 5% of the coco glucoside;
22% to 23% of the glycerin;
beeswax;
coconut oil, and
stearic acid;
wherein the respective amounts are based on weight.
